(19) United States
(12) Patent Application Publication
(10) Pub. No.: US 2020/0206443 A1
(43) Pub. Date: Jul. 2, 2020

(54) ANESTHESIA MACHINE AND AUTOMATIC VENTILATION SYSTEM AND METHOD THEREOF

(71) Applicant: General Electric Company, Schenectady, NY (US)

(72) Inventor: Qihu ZHU, Jiangsu (CN)

(21) Appl. No.: 16/724,533

(22) Filed: Dec. 23, 2019

(30) Foreign Application Priority Data
Dec. 26, 2018 (CN) 201811600975.2

(51) Int. Cl. A61M 16/00; A61M 16/01; G06F 3/0484; G06N 20/00

(52) U.S. Cl. CPC A61M 16/022; A61M 16/01; G06N 20/00; G06F 3/04847; A61M 16/0051

(57) ABSTRACT

The present application discloses an anesthesia machine, comprising a learning system and an operating system, wherein the learning system comprises a data input module that comprises an initial parameter setting data input module, a reference parameter data input module, and an adjusting parameter data input module, and a learning module that establishes a correspondence between adjusting parameter data and initial parameter setting data as well as reference parameter data; and wherein the operating system comprises an initial parameter setting module for receiving initial parameter setting according to the actual condition of a patient, a monitoring module for monitoring reference parameters, and an adjusting module for performing adjustment of adjusting parameters according to the correspondence established between adjusting parameter data and initial parameter setting data as well as reference parameter data and recorded by the learning module.

### TECHNICAL FIELD

The present invention generally relates to the field of anesthesia, and more specifically, to an automatic ventilation system and a method thereof during anesthesia.

### BACKGROUND

Most patients receiving general anesthesia require artificial respiration due to the absence of various reflexes and inhibited respiratory functions of the patients that are inherent in the human body. Thus artificial respiration in the operating room is generally provided to perform assisted ventilation by setting up an artificial airway. A ventilation mode commonly used in anesthesia machines at present is mechanical ventilation, which is a mode of performing ventilation on patients by the machines according to parameters. Mechanical ventilation is a complex subject and technology. First, commonly used parameters are set, including a tidal volume, a respiratory rate, an inspiration-to-expiration ratio, a positive end-expiratory pressure (PEEP), and so on. Then, basic parameters must be adjusted by an anesthesiologist according to a patient's condition or surgical operation needs. For example, parameters are manually adjusted and set by acquiring data such as an arterial blood gas analysis index, a cardiac function, a hemodynamics status, and avoidance of pulmonary tissue barotrauma. If improperly operated, surgical operations where anesthesia is used may produce postoperative complications, such as delayed awakening following general anesthesia, upper respiratory tract obstruction, hypoventilation, hypoxemia, postoperative hypotension, postoperative hypertension, arrhythmia, oliguria, or other symptoms.

### SUMMARY

In an embodiment, the present application discloses a learning system for automatic ventilation of an anesthesia machine, comprising a data input module that comprises an initial parameter setting data input module, a reference parameter data input module, and an adjusting parameter data input module; and a learning module establishing a correspondence between adjusting parameter data and initial parameter setting data as well as reference parameter data.

In an embodiment, the present application further discloses an anesthesia machine, comprising a learning system and an operating system, wherein the learning system comprises a data input module that comprises an initial parameter setting data input module, a reference parameter data input module, and an adjusting parameter data input module, and a learning module establishing a correspondence between adjusting parameter data and initial parameter setting data as well as reference parameter data; and wherein the operating system comprises an initial parameter setting module for receiving initial parameter setting according to the actual condition of a patient, a monitoring module for monitoring reference parameters, and an adjusting module for performing adjustment of adjusting parameters according to the correspondence established between adjusting parameter data and initial parameter setting data as well as reference parameter data and recorded by the learning module.

In an embodiment, the present application further discloses a method for operating an anesthesia machine, comprising: setting initial parameters according to the actual condition of a patient; monitoring reference parameters; comparing with the set initial parameters and the monitored reference parameters according to a correspondence between adjusting parameter data and initial parameter setting data as well as reference parameter data established by a learning module; and performing adjustment of adjusting parameters or providing an alarm according to a comparison result.

In an embodiment, the present application further discloses an automatic ventilation method, comprising: receiving initial parameter setting data input; receiving reference parameter data input; receiving adjusting parameter data input; establishing a correspondence between adjusting parameter data and initial parameter setting data as well as reference parameter data; setting initial parameters according to the actual condition of a patient; monitoring reference parameters; comparing with the set initial parameters and the monitored reference parameters according to the correspondence between adjusting parameter data and initial parameter setting data as well as reference parameter data established by a learning module; and performing adjustment of adjusting parameters or providing an alarm according to a comparison result.

### BRIEF DESCRIPTION OF THE DRAWINGS

These and other features, aspects, and advantages of the present invention will become better understood by reading the following detailed description with reference to the accompanying drawings, where similar reference signs are used to represent similar components throughout the accompanying drawings. The figures are as follows:

FIG. 1 is a schematic diagram of an anesthesia machine according to an embodiment of the present invention;

FIG. 2 is a block diagram of an automatic ventilation system according to an embodiment of the present invention;

FIG. 3 is a block diagram of a learning system in an automatic ventilation system according to an embodiment of the present invention;

FIG. 4 is a block diagram of an operating system in an automatic ventilation system according to an embodiment of the present invention;

FIG. 5 is a flowchart of an automatic ventilation learning method according to an embodiment of the present invention;

FIG. 6 is a flowchart of an automatic ventilation operation method according to an embodiment of the present invention; and

FIG. 7 is a flowchart of an automatic ventilation method according to another embodiment of the present invention.

### DETAILED DESCRIPTION

Specific embodiments of the present invention will be described in detail below with reference to the accompanying drawings in order to assist those skilled in the art with understanding the subject matter specifics claimed in the present invention. In the following detailed description of these specific embodiments, this description does not describe in detail any of the well-known functions or configurations are not described in detail so as to avoid unnecessary details that affect the disclosure of the present invention.

Unless otherwise defined, the technical or scientific terms used in the claims and the description are as they are commonly understood by those of ordinary skill in the art to which the present invention pertains. “First,” “second” and similar words used in this description and the claims do not denote any order, quantity or importance, but are merely intended to distinguish between different constituents. “One,” “a” and similar words are not meant to be limiting, but rather denote the presence of at least one. Unless otherwise indicated, “front,” “back,” “lower” and/or “upper” and similar words are used for illustrative purposes only and not limited to a location or a spatial orientation. “Or” and similar words are inclusive and cover one or all of the listed items. “Comprising,” “having” and similar words mean that the elements or articles appearing before “comprising” or “having” include the elements or articles and their equivalent elements appearing behind “comprising” or “having,” not excluding any other elements or articles. “Connect,” “connected” and similar words are not restricted to physical or mechanical connections, but may also include electrical connections or couplings, whether direct or indirect.

Embodiments of the present invention may be described in terms of functional components and various processing steps. It should be understood that such functional components may be implemented by any number of hardware, software and/or firmware components that are configured to perform specific functions. For example, the embodiments of the present invention may employ various integrated circuit components such as memory elements, digital signal processing elements, logic elements, or lookup tables which, under the control of one or a plurality of microprocessors or other control devices, can perform various functions of a “controller.” In addition, only one typical embodiment is shown for the system described here.

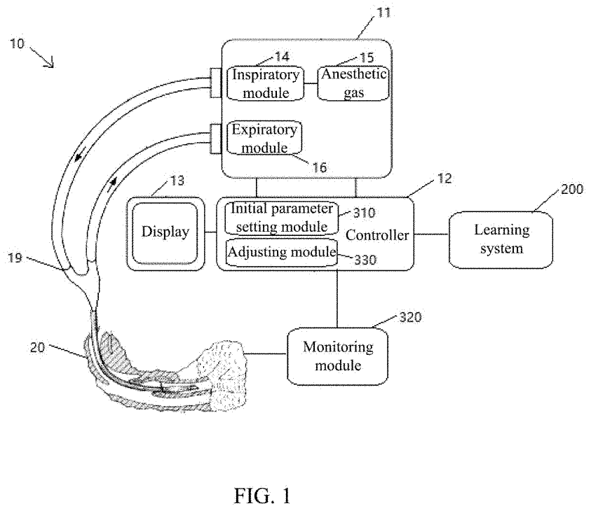

FIG. 1 shows an anesthesia machine system 10, which includes at least a pneumatic system 11 connected to a chest 20 of a patient through a pipe system 19. The pneumatic system 11 may include at least an inspiratory module 14 and an expiratory module 16. During use of anesthetic gas, the anesthetic gas 15 is in communication with the inspiratory module 14 and enters the body through the inspiratory module 14 to anesthetize the human body. The respiratory system 10 further includes a controller 12 that can set, control, or adjust parameters of the inspiratory module 14, the expiratory module 14, the anesthetic gas 15, and other modules of the pneumatic system 11. In an embodiment, the respiratory system 10 further includes a display 13 that can be used for displaying input or output of the controller 12.

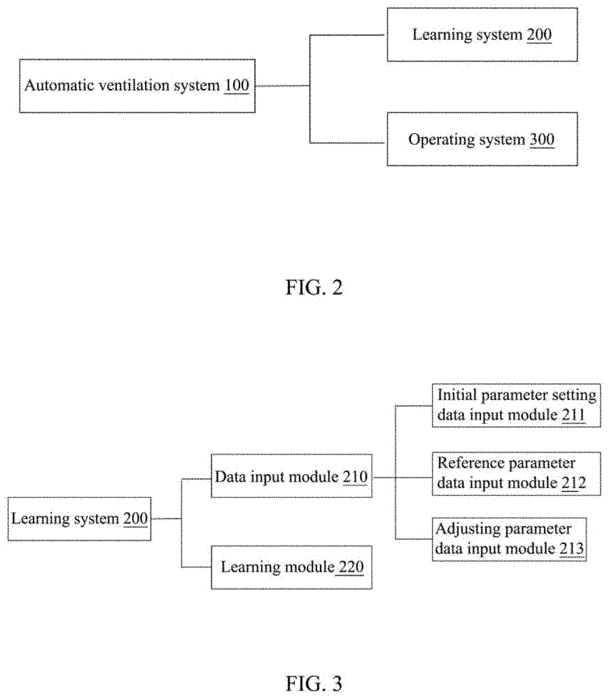

As shown in FIG. 2 and FIG. 3, the present application discloses an automatic ventilation system 100 of an anesthesia machine, which includes a learning system 200. The learning system 200 includes a data input module 210 that includes an initial parameter setting data input module 211, a reference parameter data input module 212, and an adjusting parameter data input module 213, and a learning module 220. The learning module 220 establishes a correspondence between adjusting parameter data and initial parameter setting data as well as reference parameter data. Such a correspondence is established based on a mathematical model to form a certain input/output mapping relationship. In an embodiment, the reference parameter data input module 212 receives changed data of reference parameters, and the learning module 220 establishes a correspondence between the adjusting parameter data and the initial parameter setting data as well as the changed data of reference parameters.

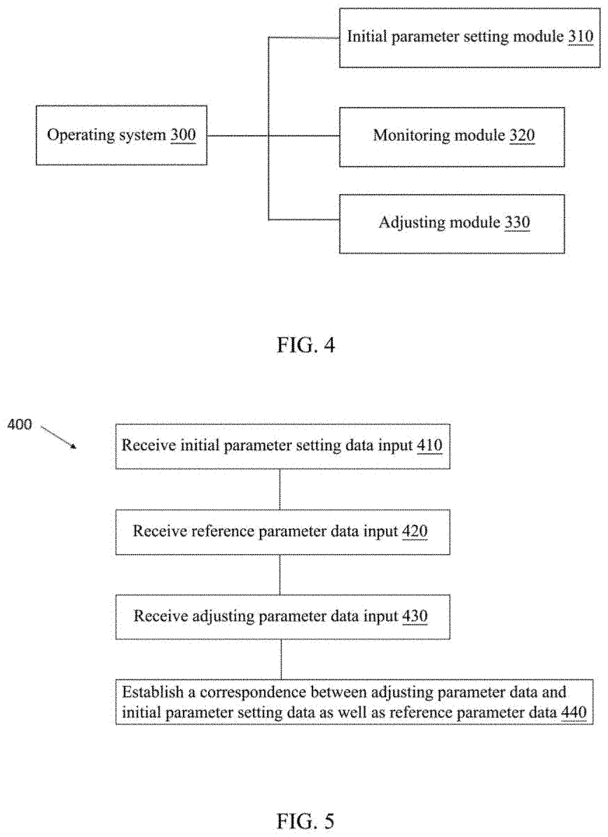

The automatic ventilation system 100 disclosed in the present application further includes an operating system 300. As shown in FIG. 4, the operating system 300 includes an initial parameter setting module 310 for receiving initial parameter setting according to the actual condition of a patient; a monitoring module 320 for monitoring reference parameters; and an adjusting module 330 for performing adjustment of adjusting parameters according to the correspondence established between adjusting parameter data and initial parameter setting data as well as reference parameter data and recorded by the learning module. In an embodiment, when the reference parameter data input module 212 receives changed data of reference parameters, the learning module 220 establishes a correspondence between the adjusting parameter data and the initial parameter setting data as well as the changed data of reference parameters, and the monitoring module 320 monitors the changed data of reference parameters.

In an embodiment, initial parameters may include basic data of the patient, such as age, weight, and gender; the initial parameters may further include data of the physical condition, such as a respiratory rate, an inspiration-to-expiration ratio, a blood pressure, a tidal volume, a plateau pressure, cardiac function data, and pulmonary compliance status rating; the initial parameters may further include pathological feature data of the patient, such as pulmonary bulla and pneumothorax; and the initial parameters may further include anesthesia data such as an anesthesia form and an anesthetic dosage.

In an embodiment, the reference parameters include an arterial blood gas analysis index, a cardiac function, a hemodynamics status, a pulmonary tissue pressure, a plateau pressure, a driving pressure, a transpulmonary pressure, a pleural pressure, surface end-expiratory pulmonary stress, an alveolar pressure, and so on.

In an embodiment, the adjusting parameters may include, for example, a respiratory rate, a positive end-expiratory pressure (PEEP), prolonged inspiratory, end-inspiratory hold and inverse ratio ventilation, and an anesthetic dose.

The adjusting parameter data corresponding to the initial parameter setting data and the reference parameter data may be obtained from a variety of sources, for example, inputted based on a doctor's historical experience; inputted based on description in related professional books; or obtained through historical record data of performing adjustment of adjusting parameters by a doctor specializing in mechanical ventilation equipment. In the case that the initial parameter setting data and the reference parameter data are the same, different adjusting parameter data may be recorded in the historical record data. In an embodiment, all data may be gathered and most-used adjusting parameter data may be selected as a final result and inputted to the adjusting parameter data input module. In another embodiment, the learning module 220 selects most-used adjusting parameter data as a final result for establishing a correspondence with the initial parameter setting data and the reference parameter data.

In still another embodiment, the learning module 220 uses the adjusting parameter data as output and the initial parameter setting data as well as the reference parameter data as input, and establishes a correspondence through a neural network in machine learning. A neural network (NN), also referred to as an artificial neural network (ANN), is a mathematical or computational model that mimics the structure and function of a biological neural network for estimating or approximating functions. The neural network performs computing using a large number of artificial neuronal connections. In most cases, the artificial neural network can change the internal structure based on external information and is a self-adaptive system. It should be noted that many types of neural networks exist, such as a full convolutional neural network and a perceptron neural network. In the embodiment of the present invention, physiological feature data may be analyzed by any one or a plurality of the above neural networks. Only some of the neural networks are disclosed in the documents of the present application. It should be understood that various neural networks generated based on the neural network principle and algorithms derived therefrom all fall within the protection scope of the present invention.

For example, in a simple example, a patient requires general anesthesia. Initial parameter data of the patient is as follows: age: 60, gender: male, weight: 70 kg, respiratory rate: a normal rate of 12 to 14 times/minute, the normal inspiration-to-expiration ratio parameter of the respiratory function set to 1:1.5, tidal volume: 6 ml/kg, plateau pressure: 30 cmH2O, and so on. During monitoring of the patient, it is detected that the arterial blood gas analysis index PaO2 as a reference parameter drops from 60 mmHg to 35 mmHg, and adjusting parameter data is reducing the inspiration-to-expiration ratio from 1:1.5 to 1:2. After the learning module establishes such a correspondence in various different cases, the learning system can be applied to perform the actual automatic ventilation operation. The automatic ventilation system 100 further includes an operating system, and an initial parameter setting module sets initial parameters according to the actual condition of the patient. For example, in an example, the initial parameters of the patient are the aforementioned initial parameter data. In one case, the monitoring module detects that the arterial blood gas analysis index PaO2 as a reference parameter drops from 60 mmHg to 35 mmHg, then the adjusting module 330 adjusts the inspiration-to-expiration ratio from 1:1.5 down to 1:2 according to the correspondence established by the learning module, namely, according to the correspondence established between adjusting parameter data and initial parameter setting data as well as reference parameter data and recorded by the learning module. In a possible embodiment, data also exists that show in the case described above, in 80% of the data, the inspiration-to-expiration ratio drops from 1:1.5 to 1:2, and 20% of the data shows that the inspiration-to-expiration ratio drops from 1:1.5 to 1:2.5. Then, most-used adjusting parameter data is selected as the final result and inputted to the adjusting parameter data input module, so that the adjusting parameter data input module 213 directly inputs inspiration-to-expiration ratio data of 1:1.5, or the learning module selects most-used adjusting parameter data as the final result for establishing a correspondence with the aforementioned initial parameter setting data and reference parameter data. In an embodiment, the learning module establishes the correspondence between adjusting parameter data and initial parameter setting data as well as reference parameter data through a neural network in machine learning. If the learning module learns that the arterial blood gas analysis index PaO2 as the reference parameter drops and thus the inspiration-to-expiration ratio needs to be lowered, for example, the PaO2 value drops from 60 mmHg to 45 mmHg, then the learning module derives that the inspiration-to-expiration ratio needs to be reduced from 1:1.5 to 1:1.8 according to the neural network in machine learning or by establishing a corresponding algorithm.

As shown in FIG. 5, the present application further discloses an automatic ventilation learning method 400, which includes the following steps: S410: receiving initial parameter setting data input; S420: receiving reference parameter data input; S430: receiving adjusting parameter data input; S440: establishing a correspondence between adjusting parameter data and initial parameter setting data as well as reference parameter data. In an embodiment, step S420 of receiving reference parameter data input includes receiving input of changed data of reference parameters, and step S440 includes establishing a correspondence between the adjusting parameter data and the initial parameter setting data as well as the changed data of reference parameters.

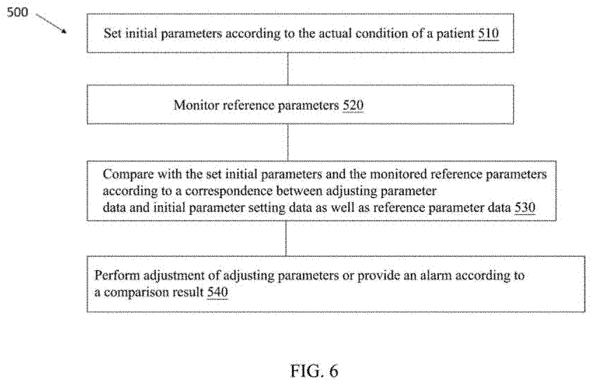

As shown in FIG. 6, the present application further discloses a method 500 for operating an anesthesia machine, which includes the following steps: S510: setting initial parameters according to the actual condition of a patient; S520: monitoring reference parameters; S530: comparing with the set initial parameters and the monitored reference parameters according to a correspondence between adjusting parameter data and initial parameter setting data as well as reference parameter data established by a learning module; and S540: performing adjustment of adjusting parameters or providing an alarm according to a comparison result. In an embodiment, the reference parameter data input module 212 is used for receiving changed data of reference parameters; the learning module 220 establishes a correspondence between the adjusting parameter data and the initial parameter setting data as well as the changed data of reference parameters; and step S520 of monitoring reference parameters includes monitoring changes of the reference parameters.

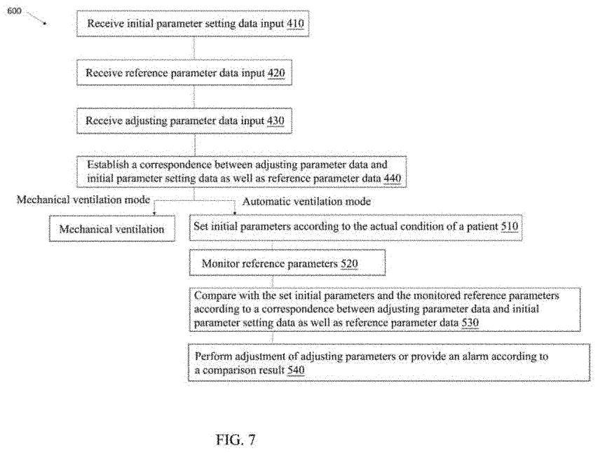

As shown in FIG. 7, the present application further discloses an automatic ventilation method 600, which includes the following steps: S410: receiving initial parameter setting data input; S420: receiving reference parameter data input; S430: receiving adjusting parameter data input; S440: establishing a correspondence between adjusting parameter data and initial parameter setting data as well as reference parameter data. In an embodiment, step S420 of receiving reference parameter data input includes receiving input of changed data of reference parameters, and step S440 includes establishing a correspondence between the adjusting parameter data and the initial parameter setting data as well as the changed data of reference parameters. In an embodiment, the automatic ventilation method further includes selecting options of an automatic ventilation mode and a mechanical ventilation mode to be selected by an operator. That is, after step S440, the operator may select to use the automatic ventilation mode or the mechanical ventilation mode. If the operator selects to use the mechanical ventilation mode, the operator sets initial parameters according to the actual condition of a patient, and then the operator manually performs adjustment of adjusting parameters according to his own judgment of how to best monitor reference parameters. If the automatic ventilation mode is selected, the following steps are further performed: S510: setting initial parameters according to the actual condition of a patient; S520: monitoring reference parameters; S530: comparing with the set initial parameters and the monitored reference parameters according to the correspondence between adjusting parameter data and initial parameter setting data as well as reference parameter data established by a learning module; and S540: performing adjustment of adjusting parameters or providing an alarm according to a comparison result. In an embodiment, step S520 of monitoring reference parameters includes monitoring changes of the reference parameters.

In an embodiment, the received initial parameter setting data input is the same as that in the aforementioned example. The patient undergoes a minimally invasive endoscopic surgery, and the reference parameter data input refers to oxygen saturation that is detected to be lower than 90%, thereby indicating that the patient is hypoxic. According to the aforementioned initial parameter setting data and reference parameter data, if the oxygen saturation is detected to be 85%, the corresponding adjusting parameter is a respiratory rate of 16 to 20 times/minute. In the case that the operator selects the mechanical ventilation mode, the operator modifies the adjusting parameters by himself according to judgment. In the case that the operator selects the automatic ventilation mode, after the operator sets the initial parameters as before according to the actual condition of the patient, the reference parameters are monitored to show that the oxygen saturation parameter monitored value drops to 85%; then comparison is performed with the set initial parameters and the monitored reference parameters according to the correspondence between adjusting parameter data and initial parameter setting data as well as reference parameter data, so as to obtain the adjusting parameter data of adjusting the respiratory rate to 16 to 20 times/minute. Accordingly, in this method, the adjusting module automatically adjusts the respiratory rate of the patient to 16 to 20 times/minute, thereby implementing the intelligent automatic adjustment function of the present application. In an embodiment, the adjusting module performs adjustment of the adjusting parameters through a neural network in machine learning according to the correspondence established between adjusting parameter data and initial parameter setting data as well as reference parameter data. If the adjusting module learns that the oxygen saturation parameter monitored value as the reference parameter drops to 88%, where the drop of the oxygen saturation parameter monitored value requires increasing the respiratory rate, then the adjusting parameter derives that the respiratory rate needs to be raised from 12 to 14 times/minute to 14 to 16 times/minute according to the neural network in machine learning or by establishing a corresponding algorithm, so as to automatically perform adjustment of the adjusting parameters.

The present application can clinically provide a patient with the optimal anesthesia and/or ventilation parameter data for use in an operation with anesthesia so as to implement or assist with implementing accurate anesthesia and/or ventilation adjustment, thereby improving efficiency before, during, and after operation and relieving suffering of the patient.

While the present invention has been described in detail with reference to the specific embodiments, it will be understood by those skilled in the art that many modifications and variations can be made to the present invention. It is, therefore, to be understood that the claims are intended to cover all such modifications and variations insofar as they are within the true spirit and scope of the present invention.

1. An automatic ventilation system of an anesthesia machine, comprising:
a data input module, comprising an initial parameter setting data input module, a reference parameter data input module, and an adjusting parameter data input module; and
a learning module establishing a correspondence between adjusting parameter data and initial parameter setting data as well as reference parameter data.

2. The automatic ventilation system of an anesthesia machine according to claim 1, wherein the reference parameter data input module receives changed data of reference parameters, and the learning module establishes a correspondence between the adjusting parameter data and the initial parameter setting data as well as the changed data of reference parameters.

3. The automatic ventilation system of an anesthesia machine according to claim 1, wherein the learning module establishes the correspondence between adjusting parameter data and initial parameter setting data as well as reference parameter data through a neural network.

4. The automatic ventilation system of an anesthesia machine according to claim 1, wherein the learning module selects most-used adjusting parameter data and establishes a correspondence between the most-used adjusting parameter data and the initial parameter setting data as well as the reference parameter data.

5. An anesthesia machine, comprising a learning system and an operating system, wherein the learning system comprises a data input module that comprises an initial parameter setting data input module, a reference parameter data input module, and an adjusting parameter data input module; and a learning module that establishes a correspondence between adjusting parameter data and initial parameter setting data as well as reference parameter data, wherein the operating system comprises: an initial parameter setting module for receiving the initial parameter setting according to the actual condition of a patient; a monitoring module for monitoring reference parameters; and an adjusting module for performing adjustment of adjusting parameters according to the correspondence established between adjusting parameter data and initial parameter setting data as well as reference parameter data and recorded by the learning module.

6. The anesthesia machine according to claim 5, wherein the reference parameter data input module receives changed data of reference parameters, the learning module establishes a correspondence between the adjusting parameter data and the initial parameter setting data as well as the changed data of reference parameters, and the monitoring module monitors the changed data of reference parameters.

7. A method for operating the anesthesia machine according to claim 5, the method comprising:
setting initial parameters according to the actual condition of a patient;
monitoring reference parameters;
comparing with the set initial parameters and the monitored reference parameters according to a correspondence between adjusting parameter data and initial parameter setting data as well as reference parameter data established by the learning module; and
performing adjustment of adjusting parameters or providing an alarm according to a comparison result.

8. The method according to claim 7, wherein the reference parameter data input module is used for receiving changed data of reference parameters, the learning module establishes a correspondence between the adjusting parameter data and the initial parameter setting data as well as the changed data of reference parameters, and the monitoring reference parameters comprises monitoring changes of the reference parameters.

9. An automatic ventilation method, comprising:
receiving initial parameter setting data input;
receiving reference parameter data input;
receiving adjusting parameter data input;
establishing a correspondence between adjusting parameter data and initial parameter setting data as well as reference parameter data;
setting initial parameters according to the actual condition of a patient;
monitoring reference parameters;
comparing with the set initial parameters and the monitored reference parameters according to the correspondence between adjusting parameter data and initial parameter setting data as well as reference parameter data established by a learning module; and
performing adjustment of adjusting parameters or providing an alarm according to a comparison result.

10. The automatic ventilation method according to claim 9, wherein the receiving reference parameter data input comprises receiving input of changed data of reference parameters, a correspondence is established between the adjusting parameter data and the initial parameter setting data as well as the changed data of reference parameters, and the monitoring reference parameters comprises monitoring changes of the reference parameters.
